(19) United States
(12) Patent Application Publication
(10) Pub. No.: US 2007/0112360 A1
(43) Pub. Date: May 17, 2007

(54) Bioprosthetic device

(76) Inventors: Patrick De Deyne, Duxbury, MA (US); Dhanuraj Shetty, Somerset, NJ (US); Steven Bowman, Sherborn, MA (US); Mark Timmer, Jersey City, NJ (US); Mora C. Melican, Cary, NC (US); Shana Azri-Meehan, Millington, NJ (US); Joseph Contiliano, Stewartsville, NJ (US)

Correspondence Address: BARNES & THORNBURG LLP, 11 SOUTH MERIDIAN, INDIANAPOLIS, IN, 46204, US

(21) Appl. No.: 11/273,745

(22) Filed: Nov. 15, 2005

(51) Int. Cl. A61F 2/02; A61F 2/08

(52) U.S. Cl. 606/151; 623/13.17; 623/23.74

(57) ABSTRACT

The present disclosure relates to bioprosthetics. For example, to the use of bioprosthetics for the repair and replacement of connective tissue.

### TECHNICAL FIELD

The present disclosure relates to bioprosthetics and particularly, for example, to the use of bioprosthetics for the repair and replacement of connective tissue.

### BACKGROUND

There are currently many ways in which various types of soft tissues, such as ligaments or tendons, for example, are reinforced and/or reconstructed. Suturing the tom or ruptured ends of the tissue is one method of attempting to restore function to the injured tissue. Sutures may also be reinforced through the use of synthetic non-bioabsorbable or bioabsorbable materials. Autografting, where tissue is taken from another site on the patient's body, is another means of soft tissue reconstruction. Yet another means of repair or reconstruction can be achieved through allograffing, where tissue from a donor of the same species is used. Still another means of repair or reconstruction of soft tissue is through xenografting in which tissue from a donor of a different species is used. Accordingly, devices and methods for the repair and replacement of connective tissue are desirable. For example, devices and methods for the repair, restoration, regeneration of spinal ligaments and spinal soft tissues are desirable.

### SUMMARY

A device or method in accordance with an illustrative embodiment of the present disclosure includes one or more of the following features or combinations thereof:

The present disclosure provides a bioprosthetic device comprising an extracellular matrix layer (hereafter extracellular matrix is referred to as ECM) and a pair of wing members. In one illustrative embodiment, the ECM layer has a body portion having an outer surface and a thickness. Each wing member extends from the body portion and has an end, a length, a outwardly facing surface and an inwardly facing surface. In this embodiment the length of each wing member is greater than the thickness of the body portion. In addition, the outwardly facing surfaces of the wing members cooperate to form an outwardly facing attachment surface extending between the ends of the wing members. In addition, the wing members may cooperate to form a V-shaped structure extending from the body portion of the ECM layer. Furthermore, the bioprosthetic device may include a synthetic reinforcement component positioned in contact with the outwardly facing attachment surface.

The device may also include at least one secondary ECM layer positioned in contact with the inwardly facing surface of a wing member and the outer surface of the body portion. The device may also include a synthetic reinforcement component positioned between the secondary ECM layer and the inwardly facing surface of a wing member. In addition, the synthetic reinforcement component may be positioned between the secondary ECM layer and the outer surface of the body portion.

In another illustrative embodiment, a bioprosthetic device is provided that comprises an ECM layer positioned in contact with a synthetic mesh reinforcement component. The density of the synthetic mesh reinforcement weave pattern is not uniform. For example, the synthetic mesh reinforcement pattern has (i) a first area with a first weave pattern, (ii) a second area with a second weave pattern and (iii) the density of the first weave pattern is greater than the density of the second weave pattern.

The bioprosthetic device may also include another synthetic mesh reinforcement component attached to the aforementioned synthetic mesh reinforcement component so that the ECM layer is interposed between both synthetic mesh reinforcement components. Each synthetic mesh reinforcement component may have a circular shape with a radius. The ECM layer may also have a circular shape with a radius. The radius of each synthetic mesh reinforcement component may be larger than the radius of ECM layer so that an outer rim portion of the each synthetic mesh reinforcement component extends beyond an edge of the ECM layer. The outer rim portion of each synthetic mesh reinforcement component can be attached so as to interpose the ECM layer.

In another illustrative embodiment a bioprosthetic device is provided that comprises an ECM layer with a pair of length-wise edges, and a pair of width-wise edges. The bioprosthetic device also includes a synthetic mesh reinforcement component wrapped around the ECM layer. The synthetic mesh reinforcement component has a weave pattern such that any angle formed by the intersection point of two fibers of the synthetic mesh reinforcement component is either acute or obtuse. The synthetic mesh reinforcement component may include a number of cross fibers which extend between length wise edges of the ECM layer and are substantially parallel to a width wise edge of the ECM layer. In addition, the device may include a pair of lateral fibers which at least extend the length of the ECM layer and are orientated relative to the ECM layer so that these fibers are substantially parallel to the length wise edges of the ECM layer.

In another illustrative embodiment of the present disclosure a bioprosthetic device is provided that includes an ECM member having a first ECM layer, a second ECM layer, a first end, and second end. A number of fibers are interposed between the first ECM layer and the second ECM layer. Each fiber has an inner portion positioned between the first and second ECM layers, and an outer portion extending outwardly from the first end or from both the first end and the second end. The inner portion inner portion of each fiber positioned between the first and second ECM layers intersects at least one other fiber so as to define either an obtuse or acute angle between the intersecting fibers.

In yet another illustrative embodiment of the present disclosure there is provided a bioprosthetic device that includes an ECM layer having a surface, a length wise edge, and a width wise edge. The device also includes at least two fiber populations both in contact with the surface of the ECM layer. Each fiber in one population is separated by a first distance. In addition, each fiber in the other population of fibers is separated by a second distance. Furthermore, the fiber populations are separated by a third distance. The third distance is greater than either the first distance or the second distance. Each fiber in each population of fibers can be positioned relative to the ECM layer so that they are substantially parallel with the width wise edge or substantially parallel with the length wise edge.

This device may also include another population of fibers placed in contact with the ECM surface. Each fiber of this population of fibers is positioned relative to the ECM layer so that they are substantially parallel with the length wise or width wise edge of the ECM layer. In addition, the fibers of this population of fibers intersects the fibers of the aforementioned populations so as to form an orthogonal angle at each intersection point.

In another illustrative embodiment of the present disclosure a prosthetic device is provided which comprises an ECM member having two ECM layers, a width wise edge, a length wise edge, and two ends. The device also includes two populations of fibers interposed between the two ECM layers. The fibers of the first population of fibers is substantially parallel with the length wise edge. These fibers have an inner portion positioned between the ECM layers and have an outer portion extending outwardly from at least one end of the ECM member. The fibers of the second population of fibers are substantially parallel with the width wise edge. Moreover, a number of fibers of the second population intersect a number of fibers of the first population so as to define an orthogonal angle.

The present disclosure also provides an illustrative embodiment of a prosthetic device which comprises an ECM member which includes a pair of ECM layers, a width wise edge, a length wise edge, and a pair of ends. The device also includes two populations of fibers interposed between the pair of ECM layers. One population is substantially parallel with the length wise edge, has an inner portion positioned between the ECM layers, and has at least one outer portion extending outwardly from an end of the ECM member. The other population of fibers is positioned between the ECM layers and are positioned relative to one another so as form a nonwoven mesh.

Additional features of the present disclosure will become apparent to those skilled in the art upon consideration of the following detailed description of embodiments exemplifying the best mode of carrying out the subject matter of the disclosure.

### BRIEF DESCRIPTION OF THE DRAWINGS

FIG. 1 is an enlarged fragmental cross sectional view of an ECM layer prior to bifurcation;

FIG. 2 is a bioprosthetic device having the ECM layer of FIG. 1 (i) after bifurcation and (ii) having a synthetic reinforcement component placed in contact with an attachment surface;

FIG. 3 an enlarged fragmental cross sectional view of an ECM layer similar to the one shown in FIG. 2 but having multiple layers;

FIG. 4 is a view similar to FIG. 3 but having a synthetic reinforce component interposed each ECM layer;

FIG. 5 is an exploded perspective view of a bioprosthetic device having an ECM layer interposed two synthetic reinforcement components;

FIG. 5A is an enlarge view of a portion of one of the synthetic reinforce components of FIG. 5;

FIG. 5B is an enlarged view of another portion of the synthetic reinforce component of FIG. 5A;

FIG. 6 is an elevafional view of the bioprosthetic device of FIG. 5, with the ECM layer positioned between the two synthetic reinforcement components;

FIG. 7 is an elevational view of a bioprosthetic wrapped in a synthetic reinforcement component;

FIG. 8 is an elevafional view of a bioprosthefic device having a number of fibers interposed two ECM layers;

FIG. 9 is a cross sectional view of the bioprosthetic device of FIG. 8 viewed in the direction indicated by arrows 9-9;

FIG. 10 is an elevational view of a bioprosthetic device in contact with a number of fibers;

FIG. 11 is an elevational view of a bioprosthetic device similar to the one shown in FIG. 10 but having the fibers orientated in a different manner;

FIG. 12 is an elevational view of a bioprosthetic device similar to the one shown in FIG. 8 but having the fibers orentated in a different manner;

FIG. 13 is a cross sectional view of the bioprosthetic device of FIG. 12 viewed in the direction indicated by arrows 13-13;

FIG. 14 is an elevational view of a bioprosthetic device similar to the one shown in FIG. 12 but having the fibers orentated in a different manner;

FIG. 15 is a cross sectional view of the bioprosthetic device of FIG. 14 viewed in the direction indicated by arrows 15-15;

FIG. 16 is an illustrative example of an embodiment of a bioprosthetic device of the present disclosure being used to repair tissue;

FIG. 17 is an illustrative example of another embodiment of a bioprosthetic device of the present disclosure being used to repair tissue;

FIG. 18 is a side view of FIG. 17; and

FIG. 19 is an illustrative example of yet another embodiment of a bioprosthetic device of the present disclosure being used to repair tissue.

### DETAILED DESCRIPTION

According to the present disclosure, a bioprosthetic device for soft tissue attachment with enhanced, reinforcement, remolding, and/or reconstruction capabilities is provided. In addition, a bioprosthetic device of the present disclosure has enhanced capabilities for the repair, restoration, regeneration of spinal ligaments and spinal soft tissues.

The device includes a layer of a naturally occurring (ECM) and a synthetic reinforcement component. For the purposes of this disclosure, it is within the definition of a naturally occurring extracellular matrix (ECM) to clean, delaminate, and/or comminute the ECM, or to cross-link the collagen fibers within the ECM. The ECM may be dehydrated or not dehydrated. However, it is not within the definition of a naturally occurring ECM to extract and purify the natural fibers and refabricate a matrix material from purified natural fibers. Compare WO 00/16822 A1. However, any other appropriate well known method of preparing ECM may be utilized in constructing a bioprosthetic device of the present disclosure.

With respect to comminuted ECM, it is contemplated that it may be positioned in contact with an ECM layer of any embodiment of a bioprosthetic device of the present disclosure. For example, comminuted ECM may be positioned between any two ECM layers of a bioprosthetic device of the present disclosure. Comminuted ECM enhances the attachment, reinforcement, remolding and/or reconstruction capabilities of the bioprosthetic device. In addition, one of ordinary skill in the art can recognize that certain embodiments of the bioprosthetic device of the present disclosure may require a biological glue between the ECM material and the synthetic reinforcement component. Comminuted ECM may also be utilized as a such a biological glue. In addition, it should be appreciated that fibrin glue or other biocompatible glues or bonding agents may also be used for this purpose.

Examples of an ECM which can be utilized, include, but are not limited to, small intestinal submucosa (hereinafter referred to as SIS), lamina propria, stratum compactum or other naturally occurring (ECM). Further, other sources of ECMs from various tissues are known to be effective for tissue remodeling as well and can be utilized in the present disclosure. These sources include, but are not limited to, stomach, bladder, alimentary, respiratory, and genital submucosa. See, e.g., U.S. Pat. Nos. 6,171,344, 6,099,567, and 5,554,389, hereby incorporated by reference. Such submucosa-derived matrices comprise highly conserved collagens, glycoproteins, proteoglycans, and glycosaminoglycans. Any appropriate ECM, or combination of ECMs, may be utilized in a bioprosthetic device of the present disclosure. With respect to SIS, porcine is widely used. However, it will be appreciated that SIS may be obtained from other animal sources, including cattle, sheep, and other warm-blooded mammals. Furthermore, a single ECM may be utilized in a bioprosthetic device of the present invention or a combination of ECMs. For example, it should be understood that an ECM mentioned anywhere in this disclosure may be made entirely from SIS or include SIS, such as a combination of SIS and another ECM.

As discussed above, the bioprosthetic device of the present disclosure may include a synthetic reinforcement component. Such a component enhances mechanical and handling properties of the bioprosthetic device. For example, a synthetic reinforcement component may function to support and maintain the desired shape of a bioprosthetic device of the present disclosure during a surgical procedure. The synthetic reinforcement component may also be utilized to, and thereby enhance, the attachment of the bioprosthetic device to a soft tissue. In addition, the synthetic reinforcement component enhances the ability of the bioprosthetic device to reinforce, reconstruct, and/or remodel a soft tissue.

The synthetic reinforcement component may be made or derived from, for example, absorbable and/or non-absorbable biocompatible materials or any combination thereof. Examples of non-absorbable biocompatible materials include silk, polyester, polyamide, polypropylene, nylon, poly(ethylene terephtalate, poly(vinylidene fluoride), and poly(vinylidene fluoride-co-hexafluoropropylene), and similar compounds.

Examples of bioresorbable materials include hydroxy acids, such as, lactic acids and glycolic acids; caprolactone; hydroxybutyrate; dioxanone; orthoesters; orthocarbonates; and aminocarbonates. Bioresorbable materials also include natural materials such as chitosan, collagen, cellulose, fibrin, hyaluronic acid; fibronectin. Additional examples of suitable biocompatible, bioabsorbable materials include, but are not limited to, aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elasfin, bioabsorbable starches, etc.) and blends thereof. Examples of aliphatic polyesters include, but are not limited to, homopolymers and copolymers of lactide (which includes lactic acid, D-,L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, χ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, 2,5-diketomorpholine, pivalolactone, χ,χ-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof. Poly(iminocarbonates), include those polymers described by Kemnitzer and Kohn, in the Handbook of Biodegradable Polymers, edited by Domb, et. al., Hardwood Academic Press, pp. 251-272 (1997) incorporated herein by reference. Copoly(ether-esters), include those copolyester-ethers as described in the Journal of Biomaterials Research, Vol. 22, pages 993-1009, 1988 by Cohn and Younes, and in Polymer Preprints (ACS Division of Polymer Chemistry), Vol. 30 (1), page 498, 1989 by Cohn (e.g. PEO/PLA) both incorporated herein by reference. Polyalkylene oxalates, include those described in U.S. Pat. Nos. 4,208,511; 4,141,087; 4,130,639; 4,140,678; 4,105,034; and 4,205,399 all of which are incorporated herein by reference. Polyphosphazenes, co-, ter- and higher order mixed monomer-based polymers made from L-lacfide, D,L-lactide, lactic acid, glycolide, glycolic acid, para-dioxanone, trimethylene carbonate and ε-caprolactone such as are described by Allcock in The Encyclopedia of Polymer Science, Vol. 13, pages 31-41, Wiley Intersciences, John Wiley & Sons, 1988 and by Vandorpe, et al in the Handbook of Biodegradable Polymers, edited by Domb, et al, Hardwood Academic Press, pp. 161-182 (1997) all of which are incorporated herein by reference. Polyanhydrides include those derived from diacids of the form HOOC—C₆H₄—O—(CH₂)ₘ—O—C₆H₄—COOH, where m is an integer in the range of from 2 to 8, and copolymers thereof with aliphatic alpha-omega diacids of up to 12 carbons. Polyoxaesters, polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213; 5,700,583; and 5,859,150 all of which are incorporated herein by reference. Polyorthoesters such as those described by Heller in Handbook of Biodegradable Polymers, edited by Domb, et al, Hardwood Academic Press, pp. 99-118 (1997) incorporated herein by reference.

Examples of structural elements synthetic reinforcement components can be made of include, but are not limited to, fibers, such as, monofilaments, sutures, yarns, or threads. Any one, or any combination of, elements may be used to construct a synthetic reinforcement component. In addition, the synthetic reinforcement component may include or be organized into, for example, a group of fibers, a braided suture, a mesh structure (which includes knitted structures), bundles of fibers, or any combination thereof. The synthetic reinforcement component may include a woven and/or or nonwoven structure. In addition, the mechanical properties of the synthetic reinforcement component can be altered by changing its density or texture.

In some embodiments, the bioprosthetic device of the present disclosure can be augmented with growth factors, peptides, amino acids, anti-microbials, analgesics, anti-inflammatory agents, anabolics, analgesics and antagonists, anaesthetics, anti-adrenergic agents, anti-asthmatics, anti-atherosclerotics, antibacterials, anticholesterolics, anti-coagulants, antidepressants, antidotes, anti-emetics, anti-epileptic drugs, anti-fibrinolytics, anti-inflammatory agents, antihypertensives, antimetabolites antimigraine agents, antimycotics, antinauseants, antineoplastics, anti-obesity agents, antiprotozoals, antipsychotics, antirheumatics, antiseptics, antivertigo agents, antivirals, appetite stimulants, bacterial vaccines, bioflavonoids, calcium channel blockers, capillary stabilizing agents, coagulants, corticosteroids, detoxifying agents for cytostatic treatment, diagnostic agents (like contrast media, radiopaque agents and radioisotopes), electrolytes, enzymes, enzyme inhibitors, ferments, ferment inhibitors, gangliosides and ganglioside derivatives, hemostatics, hormones, hormone antagonists, hypnotics, immunomodulators, immunostimulants, immunosuppressants, minerals, muscle relaxants, neuromodulators, neurotransmitters and nootropics, osmotic diuretics, parasympatholytics, para-sympathomimetics, peptides, proteins, psychostimulants, respiratory stimulants, sedatives, serum lipid reducing agents, smooth muscle relaxants, sympatholytics, sympathomimetics, vasodilators, vasoprotectives, vectors for gene therapy, viral vaccines, viruses, vitamins, oligonucleotides and derivatives, and any therapeutic agent capable of affecting the nervous system.

As used herein, the term “growth factor” encompasses any cellular product that modulates the adhesion, migration, growth, or differentiation of other cells, particularly connective tissue progenitor cells. In addition, the term “growth factor” as used herein only includes substances purposefully disposed in contact with the bioprosthetic device (e.g. disposed in contact with the ECM component) and does not include naturally occurring substances already present in contact with the device (e.g. growth factors already present n contact with the ECM component) or present in the environment the device is surgically placed.

The growth factors that may be used in accordance with the present invention include, but are not limited to, members of the fibroblast growth factor family, including acidic and basic fibroblast growth factor (FGF-1 and -2) and FGF-4, members of the platelet-derived growth factor (PDGF) family, including PDGF-AB, PDGF-BB and PDGF-AA; EGFs, members of the insulin-like growth factor (IGF) family, including IGF-I and -II; the TGF-β superfamily, including TGF-β1, 2 and 3 (including rhGDF-5), osteoid-inducing factor (OIF), angiogenin(s), endothelins, hepatocyte growth factor and keratinocyte growth factor; members of the bone morphogenetic proteins (BMP's) BMP-1, (BMP-3); BMP-2; OP-1; BMP-2A, -2B, and -7, BMP-14; HBGF-1 and -2; growth differentiation factors (GDF's), members of the hedgehog family of proteins, including indian, sonic and desert hedgehog; ADMP-1; members of the interleukin (IL) family, including IL-1 thru -6; rhGDF-5 and members of the colony-stimulating factor (CSF) family, including CSF-1, G-CSF, and GM-CSF; and isoforms thereof.

Furthermore, all of the embodiments described below have are either a rectangular or circular shape. However, it should be appreciated that any embodiment of a bioprosthetic device of the present disclosure may have any shape which is appropriate for the procedure in which it is being used. For example, the ECM component and/or the synthetic reinforcement component may be shaped as a square, a triangle, or be irregularly shaped.

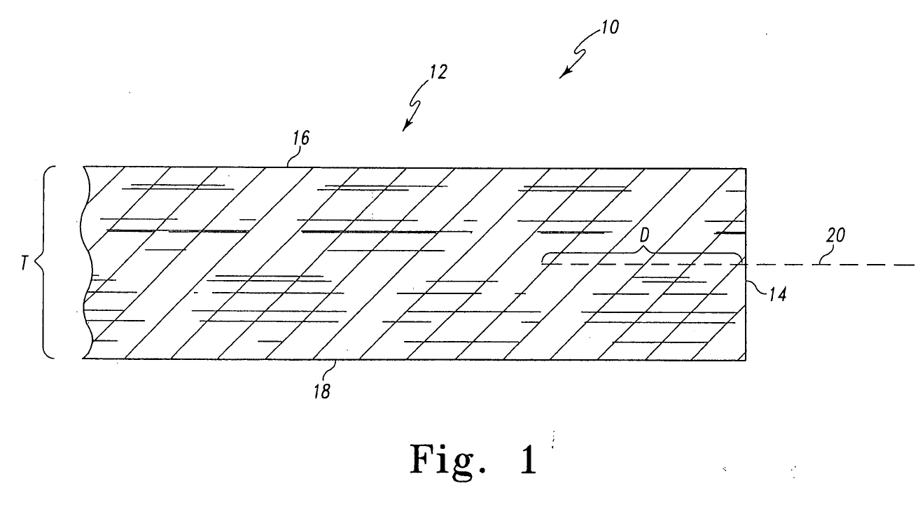

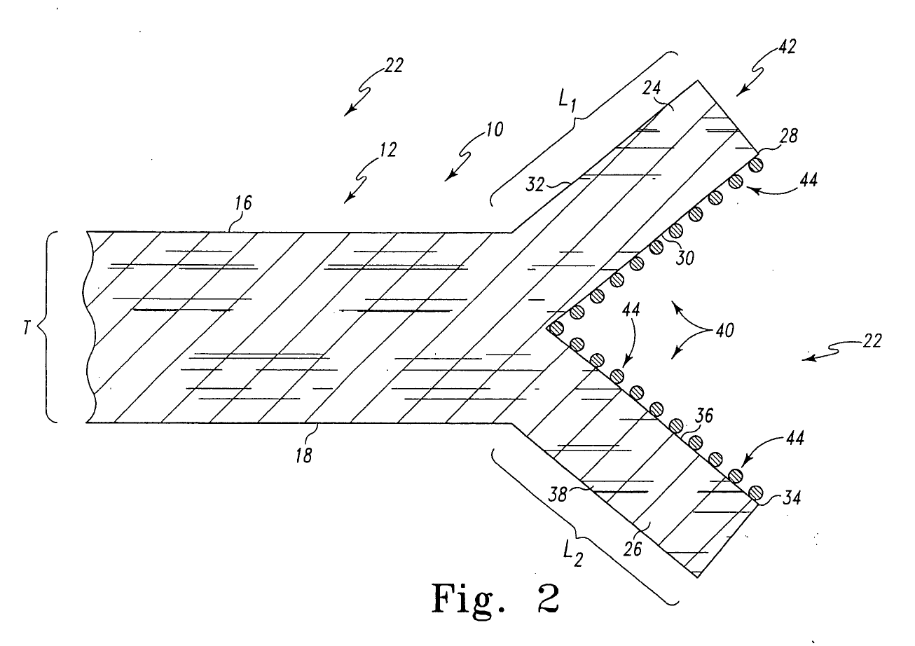

Illustrative examples of the bioprosthetic device of the present disclosure are described below. Now turning to FIGS. 1 and 2. FIG. 1 shows a layer of naturally occurring extracellular matrix 10. The ECM layer 10 has a body portion 12, an outer surface 16, an outer surface 18, an edge 14 interposed outer surfaces 16 and 18, and a thickness T. FIG. 1 illustrates a bifurcation axis 20 extending into ECM layer 10 through edge 14 and between outer surface 16 and 18. As shown in FIG. 1, ECM layer 10 is split along bifurcation axis 20 to a distance D. Preferably, distance D is greater that thickness T. The bifurcation of ECM layer 10 along bifurcation axis 20 forms one embodiment of a bioprosthetic device of the present disclosure, i.e. bioprosthetic device 22 illustrated in FIG. 2.

As shown in FIG. 2, bioprosthetic device 22 may include a pair of wing members 24 and 26 extending from body portion 12. Wing member 24 includes an end 28, a length L₁, an outwardly facing surface 30 facing away from body portion 12, and an inwardly facing surface 32 facing toward body portion 12. Wing member 26 also includes an end 34, a length L₂, an outwardly facing surface 36 facing away from body portion 12, and an inwardly facing surface 38 facing toward body portion 12. Since bifurcation axis 20 is preferably greater than thickness T, the lengths L₁ and L₂ are greater than the thickness T. In the illustrative embodiment shown in FIG. 2, wing members 24 and 26 cooperate form a V-shaped structure 42 extending from body portion 12. However, it should be understood that wing members 24 and 26 may cooperate to form other structures, for example, a T-shaped structure, or a structure where wing members 24 and 26 are pushed back to a degree so that each inwardly facing surface 32 and 38 is positioned in contact with outer surfaces 16 and 18.

In addition, as shown in FIG. 2, bifurcation of ECM layer 10 along bifurcation axis 20 results in outwardly facing surfaces 30 and 36 cooperating to form an outwardly facing attachment surface 40 extending between end 28 of wing member 24 and end 34 of wing member 26. Accordingly, having an outwardly facing attachment surface 40 increases the surface area of edge 14 (see FIG. 1) of ECM layer 10. It should be appreciated that when the bioprosthetic device is utilized in a surgical procedure, the outwardly facing attachment surface 40 may be placed in contact with a soft tissue surface, sandwiching the tissue. The increased surface area of outwardly facing attachment surface 40 enhances the ability of ECM layer 10 to attach to the desired soft tissue. In addition, as shown in FIG. 2, if desired a synthetic reinforcement component 44 may be positioned in contact with, and attached to, outwardly facing attachment surface 40. As discussed above, synthetic reinforcement component 44 may have any desired configuration as long as it performs the desired function.

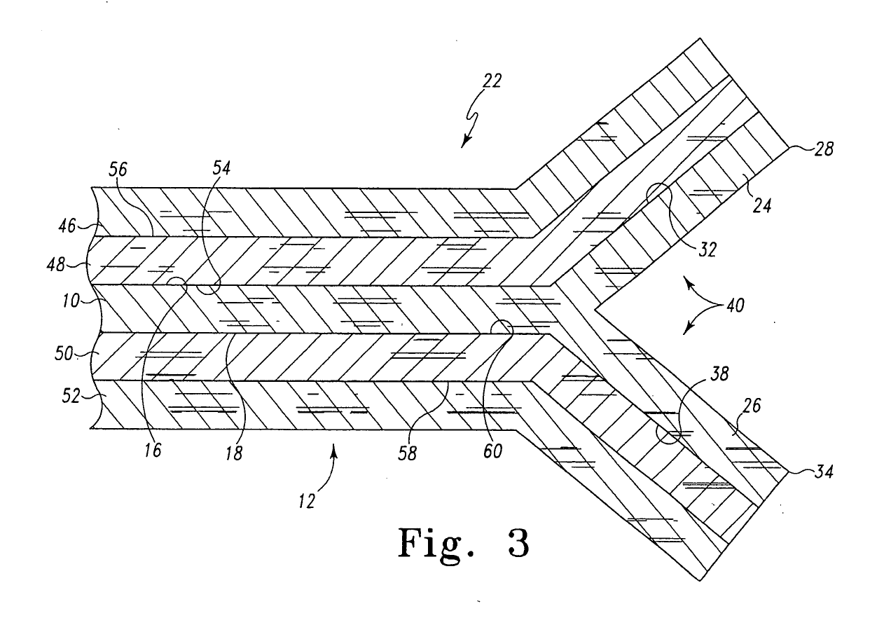

Now turning to FIG. 3, it should be appreciated that bioprosthetic device 22 may also include a number secondary ECM layers. As shown in FIG. 3, bioprosthetic device 22 includes a total of four secondary ECM layers 46, 48, 50, and 52. Each secondary layer 46, 48, 50, and 52 has a pair of exterior surfaces, however, these are only pointed out in FIG. 3 for secondary layers 48 and 50. In particular, secondary ECM layer 48 has exterior surfaces 54 and 56, and secondary ECM layer 50 has exterior surfaces 58 and 60. Secondary ECM layer 48 is positioned relative to ECM layer 10 so that the exterior surface 54 of secondary ECM layer 48 is in contact with outer surface 16 and inwardly facing surface 32 of ECM layer 10. In a similar manner, secondary ECM layer 50 is positioned relative to ECM layer 10 so that the exterior surface 60 of secondary ECM layer 50 is in contact with outer surface 18, and inwardly facing surface 38 of ECM layer 10. Still referring to FIG. 3, secondary ECM layer 46 is positioned in contact with exterior surface 56 of secondary ECM layer 48. Secondary ECM layer 52 is positioned in contact with exterior surface 58 of secondary ECM layer 50. As indicated above, comminuted ECM, may be placed between any two ECM layers of bioprosthetic device 22.

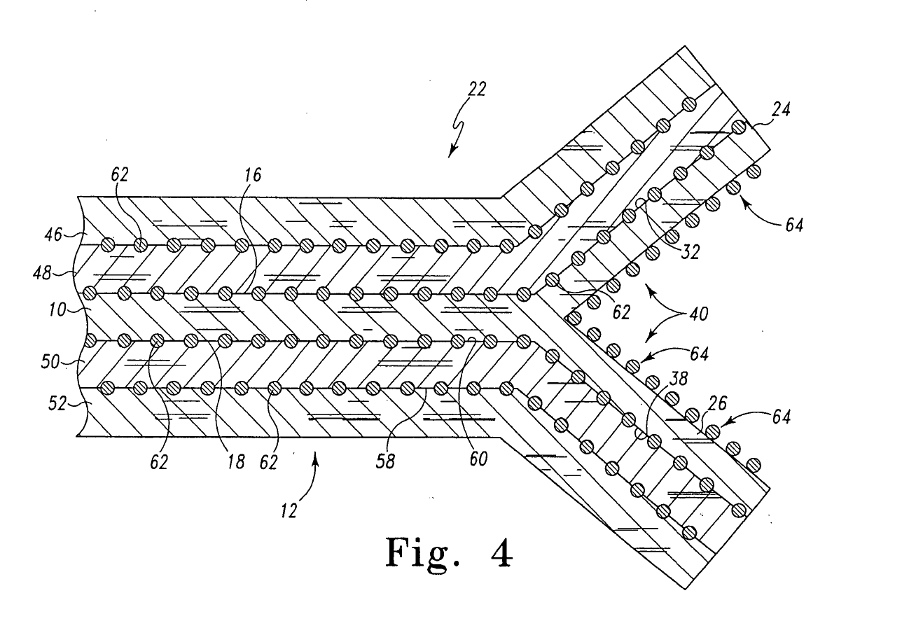

In a similar manner as shown in FIG. 2, the embodiment shown in FIG. 3 may also include synthetic reinforcement components. For example, as shown in FIG. 4 bioprosthetic device 22 may include a synthetic reinforcement component 64 positioned in contact with outwardly facing attachment surface 40 of ECM layer 10. Still referring to FIG. 4, a number of synthetic reinforcement components may be interposed ECM layer 10 and secondary ECM layers 46, 48, 50, and 52. For example, a synthetic reinforcement component 62 may be positioned interposed (i) secondary ECM layers 46 and 48, (ii) ECM layer 10 and secondary ECM layer 48, (iii) ECM layer 10 and secondary ECM layer 50, and (iv) secondary layer 50 and secondary ECM layer 52. If desired, having synthetic reinforcement component 62 positioned in the above described manner results in the reinforcement component 62 being interposed a secondary ECM layer and an inwardly facing surface of a wing member. Furthermore, it may result in having a synthetic reinforcement component interposed a secondary ECM layer and an outer surface of body portion 12.

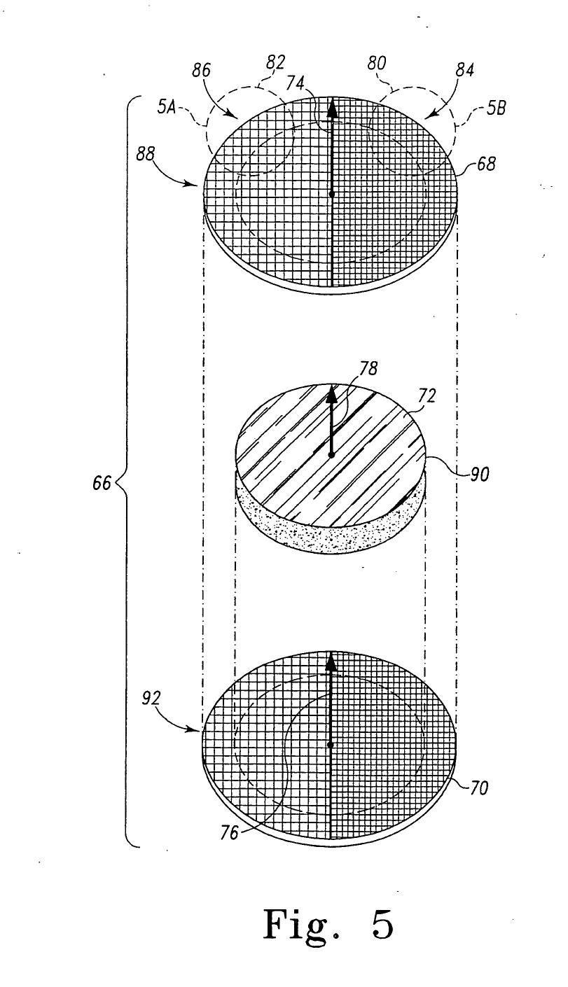

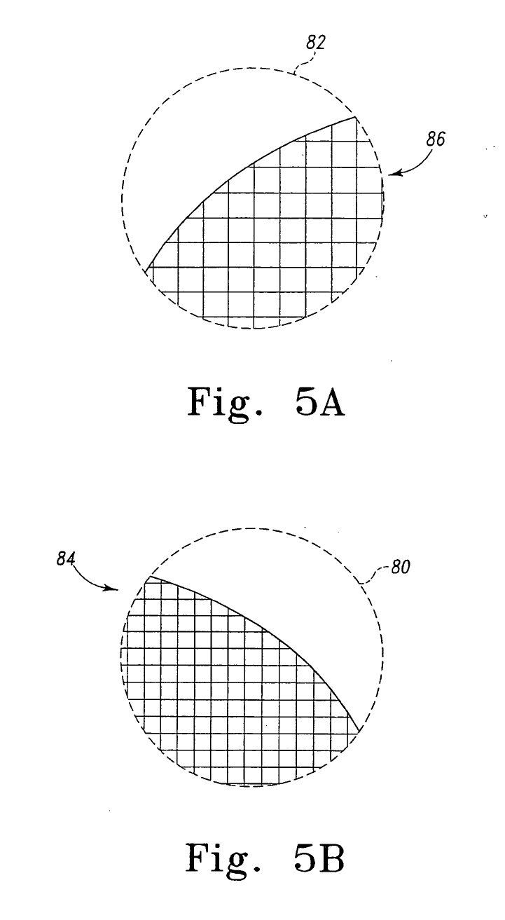

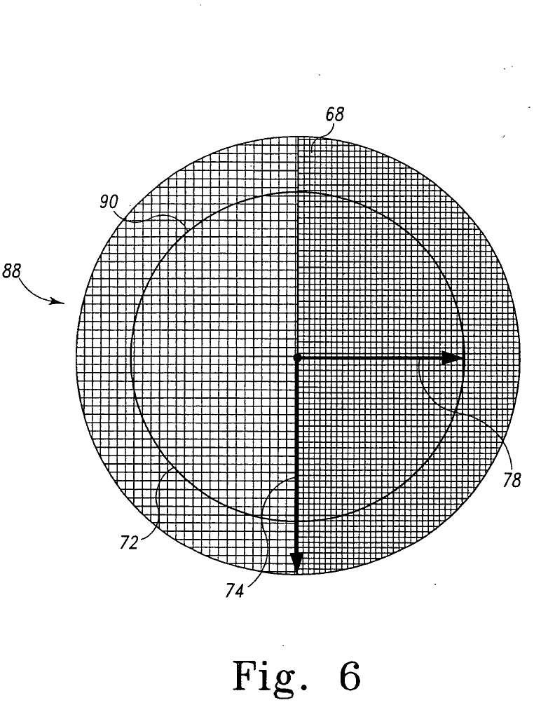

FIG. 5 illustrates another embodiment of a bioprosthetic device 66 of the present disclosure. Bioprosthetic device 66 may include synthetic mesh reinforcement components 68 and 70. In FIG. 5 both synthetic mesh reinforcement components 68 and 70 are circular in shape, however, as previously mentioned for any bioprosthetic device of the present disclosure, other shapes are contemplated, including but not limited to rectangular, square, triangle or any other geometric shape including irregular shaped components. The bioprosthetic device 66 may also include an ECM layer 72. Since the embodiment of the bioprosthetic device 66 illustrated in FIGS. 5 and 6 has a circular shape each synthetic mesh reinforcement component 68 and 70 has a radius 74 and 76, respectively. Furthermore, ECM layer 72 also has a radius 78 which is smaller than the radius 74 and 76. Synthetic mesh reinforcement component 68 includes an area 80 and an area 82. An enlarged view of area 82 is shown in FIG. 5A, while an enlarged view of area 80 is shown in FIG. 5B. Area 80 has a weave pattern 84, while area 82 has a weave pattern 86. The density of weave patterns 84 and 86 may be different. For example, the density of weave pattern 84 may be grater than the density of weave pattern 86 as shown in FIGS. 5A and 5B. In a similar manner, synthetic mesh reinforcement component 70 may also include two areas which have different weave densities.

In FIG. 5 one half of each synthetic mesh reinforcement component 68 and 70 has a weave density greater than the other half. However, it should be appreciated that any configuration of differing weave densities can be utilized as long as the weave density of the synthetic mesh reinforcement component is not uniform. Any mechanism for altering the weave density can be utilized. Examples of such mechanisms include, but are not limited to, (i) having the elements (e.g. fibers) of the synthetic mesh reinforcement component in one area closer to one another than the elements in another area, (ii) using larger elements (e.g. circumference of the fiber) in one area of the synthetic mesh reinforcement component as compared to another area, (iii) utilizing a different weave pattern in one area as compared to another area, or (iv) incorporating a different material in one area of the synthetic mesh reinforcement component as compared in another area, or any combination thereof.

As shown in FIGS. 5 and 6, synthetic mesh reinforcement component 68 may be attached to synthetic mesh reinforcement component 70 so that the ECM layer 72 is interposed synthetic mesh reinforcement component 68 and synthetic mesh reinforcement component 70. In addition, since radius 74 and 76 of synthetic mesh reinforcement components 68 and 70 may be greater than radius 78 of ECM layer 72 (i) an outer rim portion 88 of synthetic mesh reinforcement component 68 may extend beyond an edge 90 of ECM layer 72 and (ii) an outer rim portion 92 of synthetic mesh reinforcement component 70 may extend beyond edge 90 of ECM layer 72, and (iii) outer rim portion 88 of synthetic mesh reinforcement component 68 and outer rim portion 92 of synthetic mesh reinforcement component 70 may be attached so as to interpose ECM layer 72. Synthetic mesh reinforcement components 68 and 70 may be attached by any acceptable mechanism, e.g. the two components may be attached with a fiber woven therethrough, a suture, melted together (crimped) and/or a biocompatible glue or bonding agent.

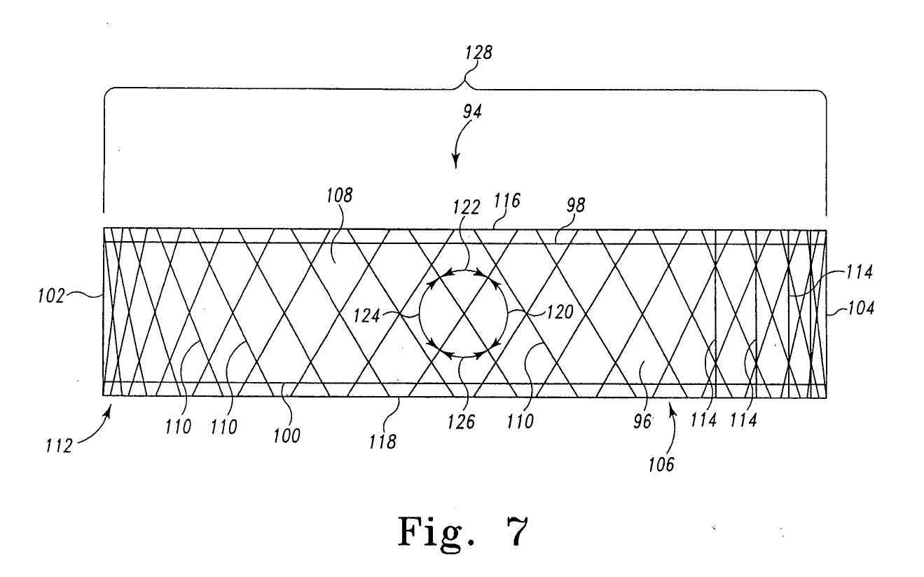

As shown in FIG. 7, another embodiment of a bioprosthetic device 94 of the present disclosure may include an ECM layer 96 having (i) a surface 108, (ii) a length 128, (iii) a pair of length wise edges 98 and 100 and (iv) a pair of width wise edges 102 and 104. Bioprosthetic device 94 may include a synthetic mesh reinforcement component 106 positioned in contact with ECM layer 96. For example, synthetic mesh reinforcement component 106 may be wrapped around ECM layer 96. As indicated, synthetic mesh reinforcement component 106 may include a number of fibers 110, cross fibers 114, and lateral fibers 116 and 118, organized into a mesh 112. The fibers 110 of the mesh 112 may be organized into a weave pattern such that the any angle formed by the intersection point of two fibers 110 of the synthetic mesh reinforcement component 106 is either acute or obtuse. For example, angles 120, 122, 124, and 126 as shown in FIG. 7. Cross fibers 114 may be positioned relative to ECM layer 96 such that they (i) extend across surface 108 and length wise edges 98 and 100 and (ii) are substantially parallel with width wise edges 102 and 104. In addition, lateral fibers 116 and 118, may be positioned relative to ECM layer 96 such that (i) they extend at least the length 128 of the of ECM layer 96 and (ii) are orientated relative to ECM layer 96 so that lateral fibers 116 and 118, are substantially parallel to length wise edges 98 and 100 of ECM layer 96.

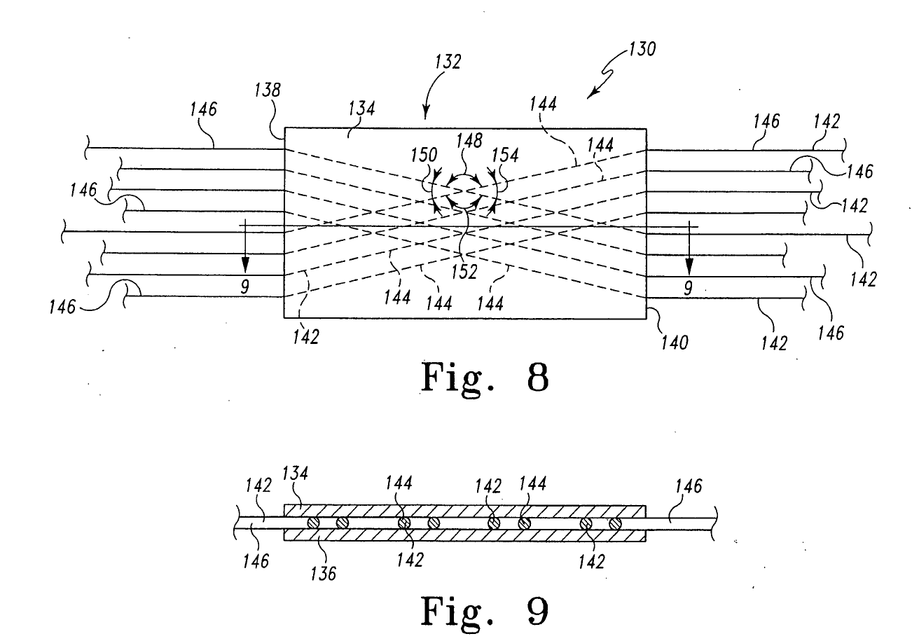

Now turning to FIG. 8 and 9, there is shown another embodiment of a bioprosthetic device 130. Device 130 may include an ECM member 132. ECM member 132 includes (i) an ECM layer 134, (ii) an ECM layer 136, and (iii) ends 138 and 140. As shown ECM layers 134 and 136 are sandwiched together. Bioprosthetic device 130 may also include a number of fibers 142 interposed ECM layers 134 and 136 as shown in FIG. 9. Each fiber 142 has (i) an inner portion positioned 144 between ECM layers 134 and 136 and (ii) at least one outer portion 146 extending outwardly from an end 138 or 140. However, as shown in FIG. 8 one or more fibers 144 may have two outer portions 146, one extending from each end 138 and 140 of bioprosthetic device 130. In addition, it should be understood that the fibers 142 are arranged relative to each other so that inner portion 144 of each fiber 144 positioned between ECM layers 134, 138 intersects at least one other inner portion 144 so as to only define obtuse or acute angles (e.g. angels 148, 150, 152, and 154) between the intersecting fibers.

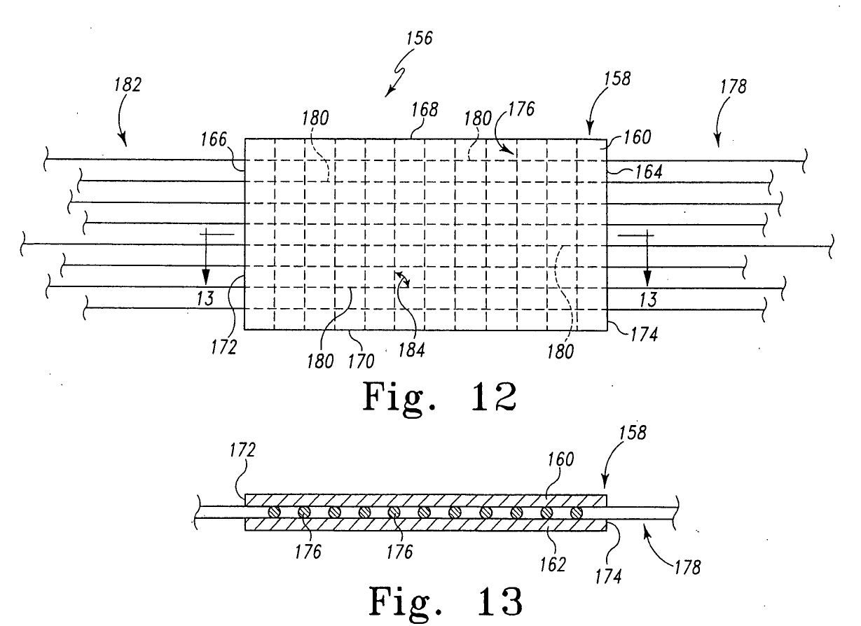

FIGS. 12 and 13 illustrate a bioprosthetic device 156 similar to device 130 shown in FIGS. 8 and 9. A bioprosthetic device 156 may include an ECM member 158 which includes (i) an ECM layer 160, (ii) an ECM layer 162, (iii) width wise edges 164 and 166, (iv) length wise edges 168 and 170, and (v) ends 172 and 174. Bioprosthetic device 156 may also include a population 176 of fibers and a population 178 of fibers interposed between ECM layers 160 and 162. With respect to population 176 and population 178 these populations are arranged relative to one another so that a number of fibers in population 178 intersects a number of fibers of population 176 so as to define an orthogonal angle 184. One of the two populations may have fibers which have an inner portion positioned between ECM layers and at least one outer portion extending outwardly from an end of an ECM member. For example, each fiber of population 178 (i) is substantially parallel with length wise edges 168 and 170, (ii) has an inner portion 180 positioned between ECM layers 160 and 162, and (iii) has at least one outer portion 182 extending outwardly from an end 172 and 174 of ECM member 158. With respect to population 176 each fiber (i) is substantially parallel with width wise edges 164 and 166, and (ii) intersects a number of fibers of population 178 so as to only define an orthogonal angle 184.

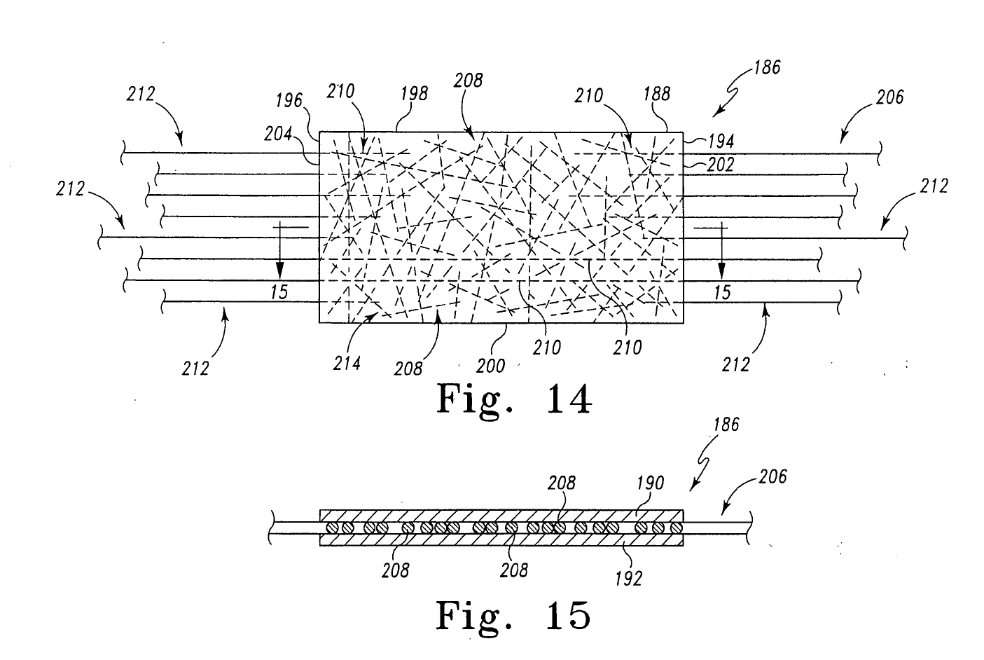

Now turning to FIGS. 14 and 15, another embodiment is illustrated. This bioprosthetic device 186 may include an ECM member 188 which includes (i) an ECM layer 190, (ii) an ECM layer 192, (iii) width wise edges 194 and 196, (iv) length wise edges 198 and 200, and (v) ends 202 and 204. A population of fibers 206 and 208 are interposed ECM layers 190 and 192. Each fiber of population 206 (i) is substantially parallel with a length wise edge 198 or 200, (ii) has an inner portion 210 positioned between ECM layers 190 and 192, and (iii) has an outer portion 212 extending outwardly from an end 202 and/or 204. With respect to population 208, the fibers are positioned relative to one another so as form a nonwoven mesh 214.

With respect to the embodiments illustrated in FIGS. 8-9 and 12-15, in each of these embodiments the ECM member is shown as a rectangle, however, as for any embodiment of the present disclosure, it should be appreciated that other shapes for the ECM member are contemplated as long as (i) the inner portions of the fibers intersect to form an acute or obtuse angle and at least one fiber has an outer portion, or (ii) two populations of fibers intersect to form an orthogonal angle and at least one fiber has an outer portion, or (iii) one population of fibers forms a nonwoven mesh and the other population has at least one fiber with an outer portion.

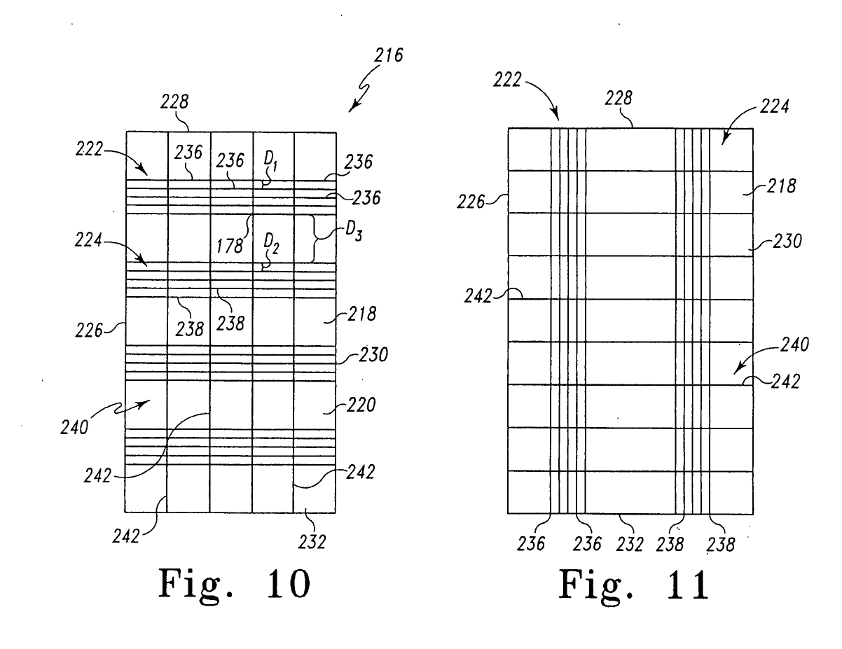

FIGS. 10 and 11 illustrate other embodiments of bioprosthetic devices of the present disclosure. In FIG. 10 bioprosthetic device 216 may include an ECM layer 218, having (i) a surface 220, (ii) length wise edges 226 and 230 and (iii) width wise edges 228 and 232. Bioprosthetic device may also include two populations 222 and 224 of fibers positioned in contact with surface 220 of ECM layer 218. As indicated in FIG. 10 (i) each fiber 236 of population 222 is separated by a distance D1, (ii) each fiber 238 of population 224 is separated by a distance D2, (iii) populations 222 and 224 are separated by a distance D3, and (iv) D3 is larger than both D1 and D2. In one configuration of bioprosthetic device 216 each fiber 236 of population 222 and each fiber 238 of population 224 is positioned relative to ECM layer 218, so that fibers 236 and 238 are substantially parallel with width wise edges 226 and 230.

Bioprosthetic device 216 may also include a population 240 of fibers 242 in contact with surface 220. Each fiber 242 of population 240 may be positioned relative to ECM layer 218, so that each fiber 242 of population 240 is substantially parallel with the length wise edges 226 and 230.

As shown in FIG. 11, populations 222 and 224 may also be positioned relative to ECM layer 218, so as to be substantially parallel with length wise edges 226 and 230. In addition, population 240 may be positioned relative to ECM layer 218, so as to be substantially parallel with width wise edges 228 and 232.

As discussed, although ECM layer 218, of bioprosthetic device 216 has a rectangular shape, any shape can be utilized as long as there are two populations of fibers positioned in contact with the surface of the ECM layer and (i) each fiber of one of the populations is separated by a distance D1, (ii) each fiber of the other population is separated by a distance D2, (iii) the populations are separated by a distance D3, and (iv) D3 is larger that both D1 and D2.

The devices disclosed herein provide better integration of the bioprosthetic device with the contiguous soft tissues. These devices also provide a more integrated and stronger fixation technique. Exemplary illustrations of utilizing some of the embodiments of the present disclosure are discussed below.

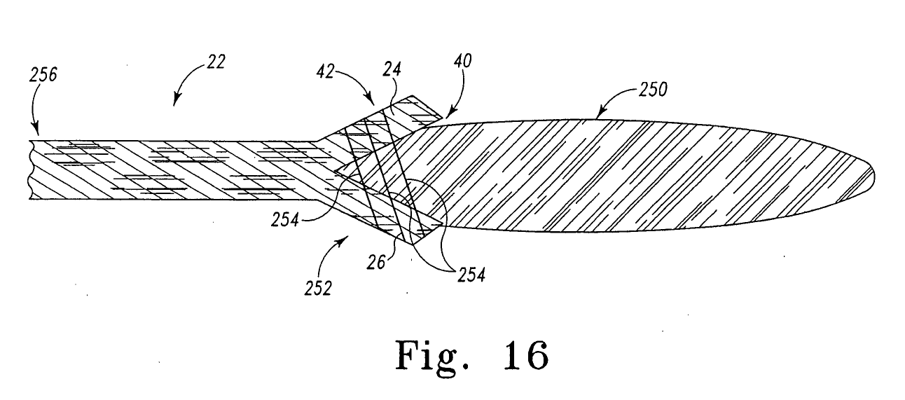

For example, FIG. 16 illustrates how bioprosthetic device 22 could be utilized in a surgical procedure to treat a repair site 252 of damaged tissue 250. In particular, as discussed above, device 22 includes wing members 24 and 26 which cooperate to form a V-shaped structure 42 and an attachment surface 40. Repair site 252 of tissue 250 is sandwiched between wing members 24 and 26 and placed in contact with attachment surface 40, while end 256 of device 22 can be directed toward the bone or tendon. As shown, multiple sutures 254 are passed through both device 22 and the tissue 250 to secure the device 22 to the tissue 250 to be repaired.

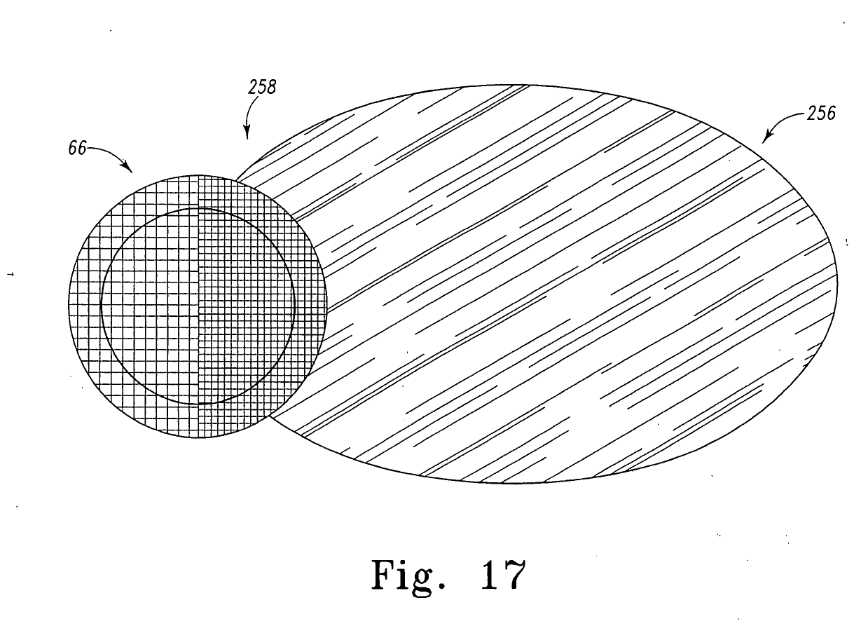

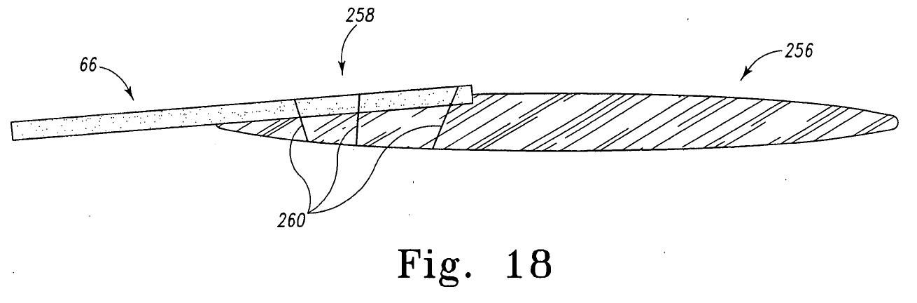

With respect to bioprosthetic device 66, FIGS. 17 and 18, show this device positioned in contact with a repair site 258 of tissue 256. In particular, circular or semi-circular-shaped tissue defects may be repaired with device 66 by covering the defect with device 66 as shown in FIGS. 17 and 18, and then passing multiple sutures 260 through both device 66 and the tissue 256.

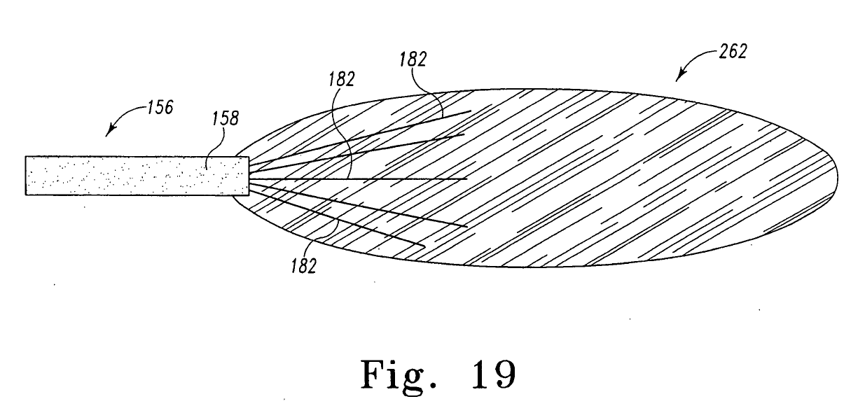

An additional use of a bioprosthetic device of the present disclosure is illustrated in FIG. 19. Here bioprosthetic device 156 is used to repair tissue 262 by inserting device 156 throughout soft tissue 262 along the longitudinal axis of force transduction. As shown, outer portions 182 of the fibers extend beyond ECM member 158 and are inserted into the tissue 262 via a needle passer paralleled with the longitudinal direction of the tissue. These outer portions 182 are then brought together by any knotting technique if so required. Note FIG. 19 only shows one set of outer portions 182 extending beyond ECM member 158, other embodiments may have more than one set as previously described in reference to FIGS. 8, 12, and 14.

While the disclosure has been illustrated and described in detail in the foregoing description, such illustration and description is to be considered as exemplary and not restrictive in character, it being understood that only the preferred embodiments have been shown and described and that all changes and modifications that come within the spirit of the disclosure are desired to be protected.

1. A bioprosthetic device, comprising:
an ECM layer having a body portion, the body portion having a thickness T;
a first wing member extending from the body portion, the first wing member having (i) an end, (ii) a length L1, and (iii) a first outwardly facing surface; and
a second wing member extending from the body portion, the second wing member having (i) an end, (ii) a length L2, and (iii) a second outwardly facing surface, wherein (i) L1 and L2 are greater that T and (ii) the first and second outwardly facing surfaces cooperate to form an outwardly facing attachment surface extending between the end of the first wing member and the end of the second wing member.

2. The bioprosthetic device of claim 1, wherein:
the ECM layer includes SIS.

3. The bioprosthetic device of claim 1, wherein:
the first wing member and the second wing member cooperate to form a V-shaped structure extending from the body portion of the ECM layer.

4. The bioprosthetic device of claim 1, further comprising:
a synthetic reinforcement component positioned in contact with the the outwardly facing attachment surface.

5. The bioprosthetic device of claim 1, further comprising:
at least one secondary ECM layer,
wherein (i) the first wing member and the second wing member each have an inwardly facing surface, (ii) the body portion has an outer surface and (iii) the secondary ECM layer is positioned in contact with the inwardly facing surface of a wing member and the outer surface of the body portion.

6. The bioprosthetic device of claim 5, further comprising:
a synthetic reinforcement component positioned between the secondary ECM layer and the inwardly facing surface of a wing member.

7. The bioprosthetic device of claim 6, wherein:
the synthetic reinforcement component is positioned between the secondary ECM layer and the outer surface of the body portion.

8. The bioprosthetic device of claim 1, further comprising:
a growth factor disposed in contact with the ECM layer.

9. A bioprosthetic device, comprising:
a first synthetic mesh reinforcement component; and
an ECM layer positioned in contact with the first synthetic mesh reinforcement component,
wherein the first synthetic mesh reinforcement component has (i) a first area with a first weave pattern, (ii) a second area with a second weave pattern and (iii) the density of the first weave pattern is greater than the density of the second weave pattern.

10. The bioprosthetic device of claim 9, wherein:
the ECM layer includes is SIS

11. The bioprosthetic device of claim 9, further comprising:
a second synthetic mesh reinforcement component having (i) a first area with a first weave pattern, (ii) a second area with a second weave pattern and (iii) the density of the first weave pattern is greater than the density of the second weave pattern,
wherein the second synthetic mesh reinforcement component is attached to the first synthetic mesh reinforcement component so that the ECM layer is interposed the first synthetic mesh reinforcement component and the second synthetic mesh reinforcement component.

12. The bioprosthetic device of claim 1 1, wherein:
the first synthetic mesh reinforcement component has a circular shape with a radius R1,
the second synthetic mesh reinforcement component has a circular shape with a radius R2,
the ECM layer has a circular shape with a radius R3,
R1 and R2 are greater than R3 so that (i) an outer rim portion of the first synthetic mesh reinforcement component extends beyond an edge of the ECM layer and (ii) an outer rim portion of the second synthetic mesh reinforcement component extends beyond the edge of the ECM layer, and
the outer rim portion of the first synthetic mesh reinforcement component and the outer rim portion of the second synthetic mesh reinforcement component are attached so as to interpose the ECM layer.

13. The bioprosthetic device of claim 9, further comprising:
a growth factor disposed in contact with the ECM layer.

14. A bioprosthetic device, comprising:
an ECM layer; and
a synthetic mesh reinforcement component having a weave pattern such that any angle formed by the intersection point of two fibers of the synthetic mesh reinforcement component is either acute or obtuse,
wherein the synthetic mesh reinforcement component is wrapped around the ECM layer.

15. The bioprosthetic device of claim 14 wherein:
the synthetic mesh reinforcement component includes a number of cross fibers; and
the cross fibers (i) extend across a first length wise edge and a second length wise edge of the ECM layer and (ii) are substantially parallel to a width wise edge.

16. The bioprosthetic device of claim 14, wherein:
the ECM layer has a length and a first length wise edge and a second length wise edge, and
the synthetic mesh reinforcement component includes a pair of lateral fibers which (i) at least extend the length L of the of the ECM layer and (ii) are orientated relative to the ECM layer so that the pair of the lateral fibers are substantially parallel to the first and second length wise edge of the ECM layer.

17. The bioprosthetic device of claim 14, wherein:
the ECM layer includes SIS.

18. The bioprosthetic device of claim 14, further comprising:
a growth factor disposed in contact with the ECM layer.

19. A bioprosthetic device, comprising:
an ECM member which includes (i) first ECM layer, (ii) a second ECM layer, (iii) a first end, and (iv) a second end;
a number of fibers interposed between the first ECM layer and the second ECM layer,
wherein (i) each fiber has an inner portion positioned between the first and second ECM layers (ii) each fiber has an outer portion extending outwardly from the first end, and (iii) the inner portion of each fiber positioned between the first and second ECM layers intersects at least one other fiber so as to define either an obtuse or acute angle between the intersecting fibers.

20. The bioprosthetic device of claim 19, wherein:
each fiber has an outer portion extending outwardly from the second end of the ECM member.

21. The bioprosthetic device of claim 19, wherein:
the ECM member includes SIS.

22. The bioprosthetic device of claim 19, further comprising:
a growth factor disposed in contact with the ECM layer.

23. A bioprosthetic device, comprising:
an ECM layer having a surface;
a first population of fibers positioned in contact with the surface; and
a second population of fibers positioned in contact with the surface,
wherein (i) each fiber of the first population of fibers is separated by a distance D1, (ii) each fiber of the second population of fibers is separated by a distance D2, (iii) the first population and the second population are separated by a distance D3, and (iv) D3 is larger that both D1 and D2.

24. The bioprosthetic device of claim 23, wherein:
the ECM layer has a length wise edge and a width wise edge, and
each fiber of the first population of fibers and each fiber of the second population of fibers is positioned relative to the ECM layer so that each fiber of the first and second populations of fibers are substantially parallel with the width wise edge.

25. The bioprosthetic device of claim 24, further comprising:
a third population of fibers placed in contact with the surface,
wherein each fiber of the third population of fibers is positioned relative to the ECM layer so that each fiber of the third population of fibers is substantially parallel with the length wise edge.

26. The bioprosthetic device of claim 23, wherein:
the ECM layer has a length wise edge and a width wise edge, and
each fiber of the first population of fibers and each fiber of the second population of fibers is positioned relative to the ECM layer so that each fiber of the first and second populations of fibers is substantially parallel with the length wise edge.

27. The bioprosthetic device of claim 26, further comprising:
a third population of fibers placed in contact with the surface,
wherein each fiber of the third population is positioned relative to the ECM layer so that each fiber of the third population of fibers is substantially parallel with the width wise edge.

28. The bioprosthetic device of claim 23, wherein:
the ECM layer includes SIS.

29. The bioprosthetic device of claim 23, further comprising:
a growth factor disposed in contact with the ECM layer.

30. A bioprosthetic device, comprising:
an ECM member which includes (i) first ECM layer, (ii) second ECM layer, (iii) a width wise edge, (iv) a length wise edge (v) a first end, and (i) a second end;
a first population of fibers and a second population of fibers interposed between the first ECM layer and the second ECM layer,
wherein each fiber of the first population of fibers (i) is substantially parallel with the length wise edge, (ii) has an inner portion positioned between the first and second ECM layers, and (iii) has an outer portion extending outwardly from the first end,
wherein each fiber of the second population of fibers (i) is substantially parallel with the width wise edge, and (ii) intersects at least one fiber of the first population of fibers so as to define an orthogonal angle.

31. The bioprosthetic device of claim 30, further comprising:
a growth factor disposed in contact with the ECM layer.

32. A bioprosthetic device, comprising:
an ECM member which includes (i) first ECM layer, (ii) second ECM layer, (iii) a width wise edge, (iv) a length wise edge (v) a first end, and (i) a second end;
a first population of fibers and a second population of fibers interposed between the first ECM layer and the second ECM layer,
wherein each fiber of the first population of fibers (i) is substantially parallel with the length wise edge, (ii) has an inner portion positioned between the first and second ECM layers, and (iii) has an outer portion extending outwardly from the first end,
wherein the fibers of the second population of fibers are positioned relative to one another so as form a nonwoven mesh.

33. The bioprosthetic device of claim 32, further comprising:
a growth factor disposed in contact with the ECM layer.
